(19) United States
(12) Patent Application Publication
(10) Pub. No.: US 2013/0345624 A1
(43) Pub. Date: Dec. 26, 2013

(54) COMPRESSION, SUCTION AND HAEMOSTATIC DELIVERY DEVICE

(71) Applicants: Vickneswaran A/L Mathenaswaran, Kuala Lumpur (MY); Alwin Kumar Rathinam, Kuala Lumpur (MY); Yuwaraj Kumar A/L Balakrishnan, Kuala Lumpur (MY); Tan Su Tung, Kuala Lumpur (MY); Norfaizal Bin Bahuri, Kuala Lumpur (MY)

(72) Inventors: Vickneswaran A/L Mathenaswaran, Kuala Lumpur (MY); Alwin Kumar Rathinam, Kuala Lumpur (MY); Yuwaraj Kumar A/L Balakrishnan, Kuala Lumpur (MY); Tan Su Tung, Kuala Lumpur (MY); Norfaizal Bin Bahuri, Kuala Lumpur (MY)

(21) Appl. No.: 13/997,359

(22) PCT Filed: May 30, 2011
(86) PCT No.: PCT/MY2011/000081

§ 371 Date: Sep. 6, 2013

(30) Foreign Application Priority Data
Dec. 23, 2010 (MY) PI2010006153

(51) Int. Cl. A61M 25/00; A61M 1/00

(52) U.S. Cl. CPC A61M 25/0026; A61M 1/0023
USPC 604/35

(57) ABSTRACT

A haemostatic agent delivery device (100) comprising an external tube (101) having a handle (102) mounted at its proximal end and an opening (104) located at its distal end for delivering a haemostatic agent over and sucking blood at a haemorrhagic site simultaneously; and an internal tube (109) having an absorbent (105) across its distal end, a controller (106) and at least one connecting means (107) at its proximal end for loading of haemostatic agent or channeling of sucking force under the trigger of the controller (106); wherein in use the internal tube (109) is introduced into the external tube (101) and pushed along the external tube (101) distally to cause the suction of blood through the absorbent (105) while simultaneously causes compression of the haemostatic agent over the haemorrhagic site.

### FIELD OF INVENTION

The present invention relates to a haemostatic agent delivery system. More particularly, the present invention provides an endoscopic and laparoscopic haemostatic agent delivery system which allows accurate and efficient delivery of a wide variety of haemostatic agent to the haemorrhagic site during surgical procedures.

### BACKGROUND OF THE INVENTION

During an endoscopic or laparoscopic surgical procedure, a surgeon would have required the use of a suction tube over a haemorrhagic site for clearing blood in order to maintain visibility. The suction tube is subsequently removed to allow the introduction of a haemostatic agent by one hand either using a forceps-like device or syringe, and almost simultaneously, another hand of the surgeon is used to place a pattie (a sponge-like material with attached string) over the haemostatic materials. This is followed by the positioning of a suction tube over the pattie to gently compress the haemorrhagic site. These series of actions require time, as the surgeon has to manoeuvre the instruments in and out of a long narrow tunnel. Therefore, the haemostatic material is usually washed away easily, especially when the bleeding is brisk.

The rapid expansion of endoscopic procedures for base of skull and intra-parenchymal lesions using tube techniques also presents surgeons with haemostasis problems. Conventional bipolar forceps are restricted in their use by poor access for delivery of haemostatic materials, such as Avitene®, Surgicel® and FloSeal®, and are often clumsy and difficult to use. The limitations in the haemostatic agent delivery device and system also affect the efficiency of other surgical procedures, especially neurosurgical procedures. Therefore, it is desirable for the industry to provide an innovative delivering system which can be applied in various surgical procedures to efficiently overcome the haemostasis problems.

There are a few patented technologies disclosed in the prior art relating to haemostatic agent delivery systems. Canadian Patent No. CA2101304 discloses an endoscopic tool for dispensing units of haemostatic agent broadly, which comprises a hollow tube that holds a plurality of individual units of the haemostatic agent, a valve at the distal end of the hollow tube which permits the haemostatic agent to pass therethrough but prevents foreign matter from contacting the haemostatic agent while in the tube, a plunger which extends into the hollow tube and contacts a proximal unit of the haemostatic agent, and a mechanism for moving the plunger distally in incremental movements to cause, upon each incremental movement, an individual unit of the haemostatic agent to be pushed through the valve. In this invention, a rachet type mechanism is utilized for delivering individual pieces of haemostatic agent, such as collagen, to a surgical site.

Another endoscopic material delivery device is also disclosed in U.S. Pat. No. 5,415,631. This patent discloses a device for delivering a quantity of material endoscopically to an intended site of application within a body through an incision or entrance wound. The device includes a handle portion with a movable trigger means and an endoscopic portion. A reservoir or cartridge containing material for delivery may be disposed in the handle or endoscopic portion of the delivery device. The operation mechanism of this delivery device is similar to the endoscopic tool of CA2101304. The trigger means can be operated in such a manner so as to cause a discharge of a metered quantity of material from the proximal end of the endoscopic tube.

European Patent No. EP1064882 also relates to a device for guided percutaneous application of a retractile staple or a haemostatic material using a novel insertion system that opens upon withdrawal, that enables operating cardiologists and endoscopic surgeons to easily, rapidly and reliably achieve haemostasis in the puncture site. This device is characterized by a perforated staple with an orifice in its center that allows to be threaded through the guide and to be guided to the puncture site, and an applicator that holds the staple or haemostatic material, allows for the percutaneous movement of said staple to the puncture site, and its activation.

There is also a laparoscopic haemostat delivery system disclosed in U.S. Pat. No. 5,310,407. This document discloses a delivery system and a method for inserting haemostatic material through a channel of a laparoscopic cannula, and for directly applying the material to an internal tissue site, includes a hollow sheath having a cross-section and configuration that permits sliding passage thereof through the channel of the laparoscopic cannula. The haemostatic material can be advanced through the lumen of the sheath and mechanically applied at the tissue site by an applicator.

Another device, system and method for improving delivery of haemostatic material are disclosed in U.S. Pat. No. 6,544,236. The device includes a vent cap body capable of removably engaging a cannula. The vent cap has a passage extending through the vent cap body from the cannula to the exterior. In one embodiment, the vent cap has a valve which has an opened and closed position to allow a gas or fluid to pass through the vent cap. The vent cap is designed to help maintain the continuity of the haemostatic material during delivery of the material from the adaptor to the trial staging chamber.

Various types of patented technologies have been disclosed in the prior art. However, none of the existing technologies is related to a device or system for delivering haemostatic agent which is capable of providing both compression and suction effects at the haemorrhagic site simultaneously. Clearly there is a need for the present invention to provide an innovative haemostatic agent delivering device or system which can efficiently overcome the drawbacks of the prior art.

### SUMMARY OF INVENTION

The primary object of the present invention is to provide a haemostatic agent delivery system which allows the delivery of haemostatic materials over the site of haemorrhage accurately and efficiently.

Another object of the present invention is to provide a haemostatic agent delivery device or system which allows both compression and suction mechanisms to be performed at the haemorrhagic site simultaneously to effectively achieve the haemostasis effect.

Still another object of the present invention is to provide a haemostatic agent delivery system which is condensed into a single device by which the surgical process can be performed more easily, even by using only one hand, thereby making delivery more efficient.

Yet another object of the present invention is to prevent wastage of haemostatic agents from being left along the internal part of the commercial applicator tube or being washed away by brisk haemorrhaging during the endoscopic or laparoscopic works.

At least one of the preceding objects is met, in whole or in part, by the present invention, in which one of the embodiments of the present invention describes a haemostatic agent delivery device (100) comprising an external tube (101) having a handle (102) mounted at its proximal end and an opening (104) located at its distal end for delivering a haemostatic agent over and sucking blood at a haemorrhagic site simultaneously; and an internal tube (109) having an absorbent (105) across its distal end, a controller (106) and at least one connecting means (107) at its proximal end for loading of haemostatic agent or channeling of sucking force under the trigger of the controller (106); wherein in use, the internal tube (109) is introduced into the external tube (101) and pushed along the external tube (101) distally to cause the suction of blood through the absorbent (105) while simultaneously causes compression of the haemostatic agent over the haemorrhagic site.

One of the preferred embodiments of the present invention discloses that the handle (102) mounted on the external tube (101) has an index finger rest (102a) and a middle finger rest (102b); whereas the controller (106) mounted on the internal tube (109) is a thumb rest.

Another preferred embodiment of the present invention discloses that the absorbent (105) is a surgical pattie or a semi-permeable membrane.

In still another preferred embodiment of the present invention, the internal tube (109) has two connecting means (107) separately extended from its proximal end for loading of haemostatic agent and channeling of sucking force. Preferably, the connecting means (107) is fabricated into an angle of inclination. A switch (110) is preferably mounted on one of the connecting means (107) for turning off the channeling of sucking force and allowing the flow of a desired solution into the device (100).

Yet another preferred embodiment of the present invention is a haemostatic agent delivery device (100) which further comprises at least one notch (108) formed at the distal end of the internal tube (109) for facilitating the loading of haemostatic agent.

The present delivery system can be applied for the delivery of a variety of surgical haemostatic agents. The delivery device provided is especially useful in all forms of endoscopic or laparoscopic procedures, in which access is usually limited and the operations occur down a long narrow tunnel. With the innovative incorporation of an absorbent, such as a surgical pattie, and the design of the device's operation system which is able to maintain both compression and suction simultaneously during the surgical procedures, the haemostatic effect at the haemorrhagic site can therefore be effectively and efficiently achieved. In addition, smaller amounts of the expensive haemostatic agents are required as the delivery device is capable of preventing wastage of materials.

One skilled in the art will readily appreciate that the present invention is well adapted to carry out the objects and obtain the ends and advantages mentioned, as well as those inherent therein. The embodiments described herein are not intended as limitations on the scope of the invention.

### BRIEF DESCRIPTION OF THE DRAWINGS

For the purpose of facilitating an understanding of the invention, there is illustrated in the accompanying drawing the preferred embodiments from an inspection of which when considered in connection with the following description, the invention, it's construction and operation and many of its advantages would be readily understood and appreciated.

FIG. 1 is a front view of the haemostatic agent delivery device (100) as described in one of the preferred embodiments of the present invention.

FIG. 2 shows the external tube (101) of the haemostatic agent delivery device (100) of FIG. 1.

FIG. 3(a) is the internal tube (109) of the haemostatic agent delivery device (100) proximal end for both loading of haemostatic anent and channeling of sucking force.

FIG. 3(b) is the internal tube (109) of the haemostatic agent delivery device (100) of FIG. 1, showing two connecting means (107) separately extended from its proximal end for loading of haemostatic agent and channeling of sucking force, respectively.

### DETAILED DESCRIPTION OF THE INVENTION

The present invention relates to a haemostatic agent delivery system. More particularly, the present invention provides an endoscopic and laparoscopic haemostatic agent delivery system which allows accurate and efficient delivery of a wide variety of haemostatic agent to the haemorrhagic site during surgical procedures.

Hereinafter, the invention shall be described according to the preferred embodiments of the present invention and by referring to the accompanying description and drawings. However, it is to be understood that limiting the description to the preferred embodiments of the invention and to the drawings is merely to facilitate discussion of the present invention and it is envisioned that those skilled in the art may devise various modifications without departing from the scope of the appended claim.

The present invention discloses a haemostatic agent delivery device (100) comprising an external tube (101) having a handle (102) mounted at its proximal end and an opening (104) located at its distal end for delivering a haemostatic agent over and sucking blood at a haemorrhagic site simultaneously; and an internal tube (109) having an absorbent (105) across its distal end, a controller (106) and at least one connecting means (107) at its proximal end for loading of haemostatic agent or channeling of sucking force under the trigger of the controller (106); wherein in use, the internal tube (109) is introduced into the external tube (101) and pushed along the external tube (101) distally to cause the suction of blood through the absorbent (105) while simultaneously causes compression of the haemostatic agent over the haemorrhagic site.

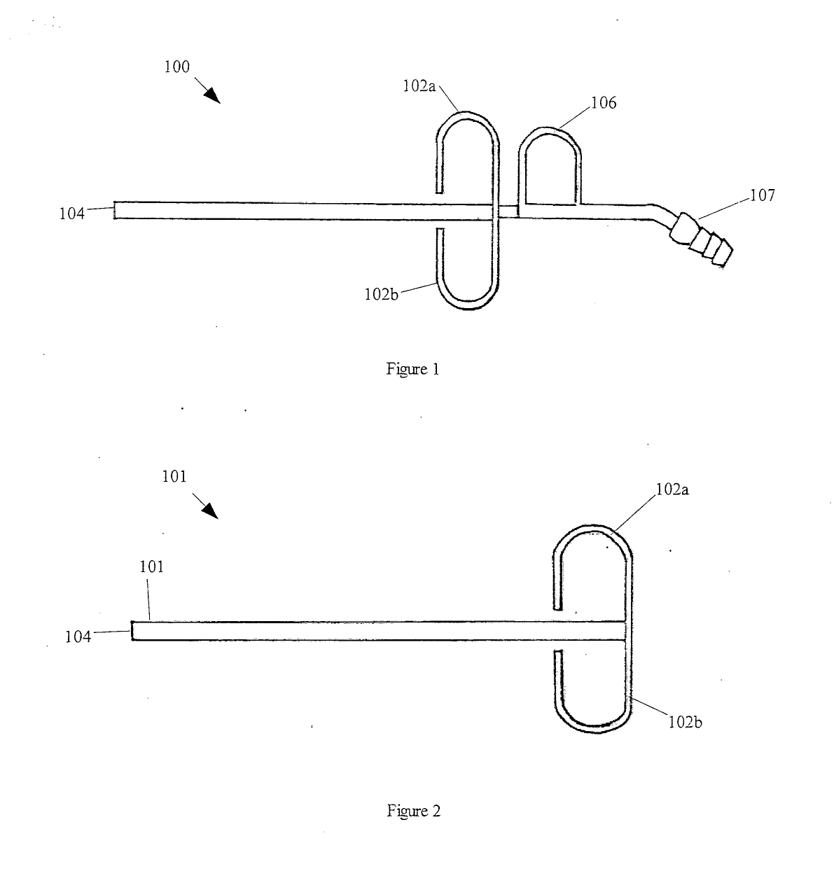

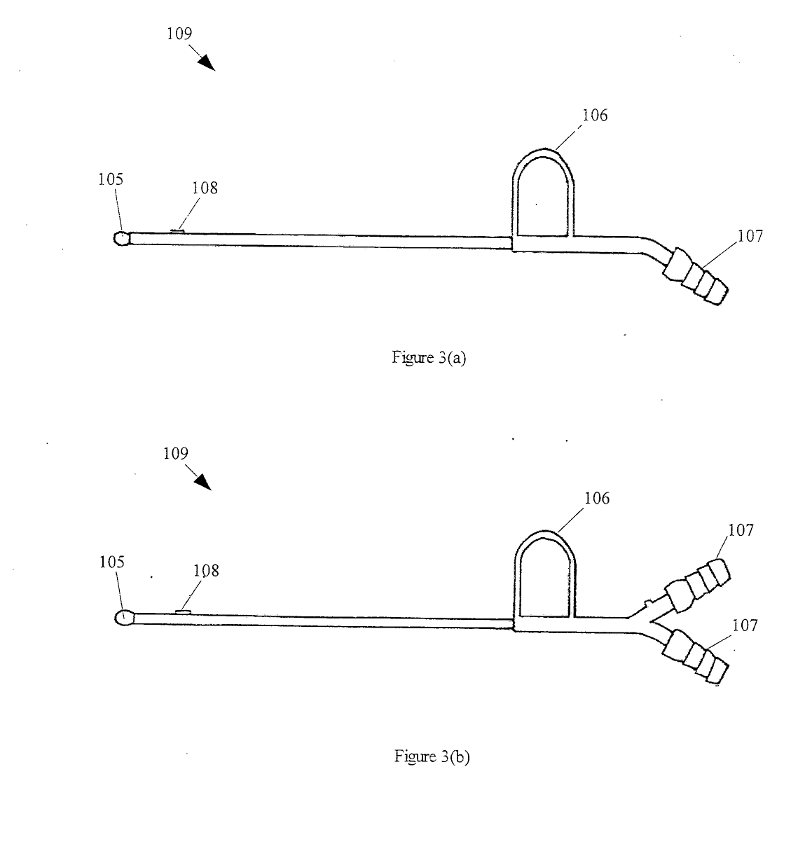

As illustrated in FIG. 1, the haemostatic agent delivery device (100) is made up of an external tube (101) and an internal tube (109). The external tube (101) is further illustrated in FIG. 2 whereas two variants of the internal tube (109) are further illustrated in FIGS. 3(a) and 3(b).

According to the preferred embodiment of the present invention, the external tube (101) as shown in FIG. 2 can be fabricated into a variety of diameters and lengths, designed to suit the different types of operation.

According to another preferred embodiment of the present invention, the external tube (101) comprises a handle mounted at its proximal end. As illustrated in FIG. 2, the handle (102) mounted on the external tube (101) is made up of an index finger rest (102a) and a middle finger rest (102b). Preferably, the handle (102) is fabricated into a C-shape in order to form the index finger rest (102a) and the middle finger rest (102b), as shown in FIG. 2.

FIGS. 3(a) and 3(b) show two different variants of the internal tube (109) according to the preferred embodiment of the present invention. The variant shown in FIG. 3(a) is an internal tube (109) to be introduced into the external tube (101) of the haemostatic agent delivery device (100) which is having only one connecting means (107) extended from its proximal end for both the mechanism of loading of haemostatic agent and channeling of sucking force. According to another embodiment of the present invention, the internal tube (109) can also comprise two connecting means (107) separately extended from its proximal end, respectively for the mechanism of loading of haemostatic agent and channeling of sucking force, as shown in FIG. 3(b). In a more preferred embodiment of the present invention, a switch (110) can be provided and preferably mounted on one of the connecting means (107) for turning off the channeling of sucking force and allowing the flow of a desired solution into the device (100).

Preferably, the connecting means (107) is fabricated into a desired angle of inclination, depending on its design and application. When more than one connecting means (107) is fabricated, these connecting means (107) shall be formed to incline towards different directions in order to facilitate connection of the device (100) to different types of input.

As shown in FIGS. 3(a) and 3(b), there is a controller (106) mounted on the internal tube (109), nearer to its proximal end. Preferably, the controller (106) is a thumb rest, which is preferably formed into a U-shape in order to accommodate the thumb. As shown in FIG. 1, the internal tube (109) is fabricated to have a smaller diameter as compared to the external tube (101) in order for it to be inserted into the external tube (101). Besides, the internal tube (109) is longer than the external tube (101). Therefore, the internal tube (109) of the haemostatic agent delivery device (100) can be introduced into the external tube (101) of the device (100) until the controller (106) mounted on the internal tube (109) meets with the handle (102) mounted on the proximal end of the external tube (101). Such a design allows the functionality of the device (100) upon the finger movements of a surgeon around the handle (102) and the controller (106), thus allowing a surgeon to control the device (100) easily using even a single hand. The device (100) is user-friendly and designed for either left-handed and right-handed user.

In another preferred embodiment of the present invention, an absorbent (105) is provided across the distal end of the internal tube (109). The adsorbent (105) is preferably a surgical pattie which facilitates the mechanism of blood suction to achieve the haemostatic effect. Alternatively, The surgical pattie can also be replaced by a semi-permeable membrane that can be directly attached to a sucker. This membrane shall not be permeable to the haemostatic agent but will be able to allow suction to be applied to absorb blood that passes through the haemostatic agent.

In yet another preferred embodiment of the present invention, the haemostatic agent delivery device (100) further comprises at least one notch (108) formed at the distal end of the internal tube (109) for facilitating the loading as well as filling of the haemostatic agent. The notches can prevent the plunger portion at the opening (104) from accidentally moving forward prior to final deployment of haemostatic agent.

The haemostatic agent delivery device (100) of the present invention can be made of metals, such as surgical steels, or plastics.

The delivery of a haemostatic agent over and sucking blood at a haemorrhagic site can be conducted simultaneously using the haemostatic agent delivery device (100) of the present invention. The process is initiated by introducing an internal tube (109) of a haemostatic agent delivery device (100) into an external tube (101) of the device (100) until a controller (106) mounted on the internal tube (109) meets a handle (102) mounted on the proximal end of the external tube (101); loading the haemostatic agent or channeling a sucking force from at least one connecting means (107) from the proximal end of the internal tube (109) into the external tube (101), under the trigger of the controller (106); pushing the internal tube (109) along the external tube (101) distally to drive the suction of blood through an absorbent (105) mounted across the distal end of the internal tube (109), and the compression of the haemostatic agent over the haemorrhagic site through an opening (104) located at the distal end of the external tube (101).

As set forth in the preceding description, the external tube (101) of the haemostatic agent delivery device (100) comes in a variety of diameters, whereas the internal tube (109) functions as a plunger or syringe. The internal tube (109) with the absorbent (105), which is preferably a pattie, across its tip or distal end, is introduced into the external tube (101). Once the tip or distal end of the internal tube (109) is almost at the distal end of the external tube (101), which is at the point where the controller (106) of the internal tube (109) meets the handle (102) of the external tube (101), the system is loaded by introducing or injecting the haemostatic agent into the external tube (101) from the distal end into the surgical or haemorrhagic site.

When the system is introduced into the operative field, suction across the absorbent (105), preferably the pattie, ensures that the blood will be sucked into the external tube (101) past the haemostatic agent and pattie into the internal tube (109). This mechanism allows the blood to mix with and activate the haemostatic agent. In addition, the internal tube (109) also protects the haemostatic agent and prevents it from being washed away if haemorrhaging is brisk.

By using the present method, the use of sucker that is normally applied to keep the field clear of blood can be eliminated, and the sucking force can be directly channeled to the present device (100) to control bleeding during a surgical procedure. The internal tube (109) can be pushed forward like a piston driving the haemostatic agent and the backing pattie out, thereby compressing the haemostatic agent over the haemorrhagic site. Therefore, the haemostatic effect can be achieved more accurately and efficiently.

When the variant of the internal tube (109), as shown in FIG. 3(b), is applied, the loading of haemostatic agent and channeling of sucking force can be separately performed by two different connecting means (107), respectively. Preferably, the channeling of sucking force is turned off by a switch (110) mounted on one of the connecting means (107) to allow the flow of a desired solution into the device (100). This desired solution can be saline, which can be used to flush down the suction tube.

Preferably, the loading of the haemostatic agent is facilitated by at least one notch (108) formed at the distal end of the internal tube (109). The notches (108) can fix the internal tube (109) preventing it from haemostatic agent accidentally during loading and approaching the haemorrhagic site.

The present device (100) is suitable for use in neurosurgical procedures including endoscopy and laparoscopy. The method as set fort in the preceding description has been tested in a variety of cases where brisk haemorrhaging is encountered. These cases include sinus or arterial haemorrhaging during endoscopic pituitary and other anterior skull base endoscopic surgery, as well as intra-parenchymal arterial bleeding during excision of intra-parenchymal lesions using an expandable port in conjunction with an endoscope.

The present method and device is suitably used for delivering various types of haemostatic agents. For example, in the case of sinus haemorrhage, the bleeding can be easily stopped by delivering the commercially obtained Surgicel® or FloSeal® using the present method and device; whereas in the case of intra-parenchymal lesions, the commercially available FloSeal®, is used as the haemostatic agent. As shown by the evaluation tests, a smaller amount or volume of haemostatic agent is required when the present method and device is applied, because the haemostatic agent can be applied more effectively. The haemostatic material can be directly delivered onto the haemorrhagic site, thereby preventing the loss of large quantity of the haemostatic material within the tube. Besides, the haemostatic agent would also not be washed away by the rapid flow of blood.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

1. A haemostatic agent delivery device, comprising:
an external tube having a handle mounted at its proximal end and an opening located at its distal end for delivering a haemostatic agent over and sucking blood at a haemorrhagic site simultaneously; and
an internal tube having an absorbent across its distal end, a controller and at least one connecting member at its proximal end for loading of haemostatic agent or channeling of sucking force under the trigger of the controller;
wherein in use, the internal tube is introduced into the external tube and pushed along the external tube distally to cause the suction of blood through the absorbent while simultaneously causes compression of the haemostatic agent over the haemorrhagic site.

2. The device according to claim 1, wherein the handle has an index finger rest and a middle finger rest.

3. The device according to claim 1, wherein the absorbent is a surgical pattie or a semi-permeable membrane.

4. The device according to claim 1, wherein the controller is a thumb rest.

5. The device according to claim 1, wherein the internal tube has two connecting members separately extended from its proximal end for loading of the haemostatic agent and channeling of sucking force.

6. The device according to claim 5, further comprising a switch mounted on one of the connecting members for turning off the channeling of sucking force and allowing the flow of a desired solution into the device.

7. The device according to claim 1, further comprising at least one notch formed at the distal end of the internal tube for facilitating the loading of the haemostatic agent.
